(19) United States
(12) Patent Application Publication
(10) Pub. No.: US 2023/0172966 A1
(43) Pub. Date: Jun. 8, 2023

(54) COMPOSITION FOR THE PREVENTION AND TREATMENT OF FOLATE AND/OR VITAMIN B12 DEFICIENCY CONDITIONS, PARTICULARLY HYPERHOMOCYSTEINEMIA

(71) Applicant: INPHA RESEARCH S.R.L., Milano (IT)

(72) Inventors: Andrea Fratter, Olmo di Martellago (VE) (IT); Simone Castelli, Milano (IT); Giuseppe Samaritani, Milano (IT)

(73) Assignee: INPHA RESEARCH S.R.L., Milano (IT)

(21) Appl. No.: 17/997,833

(22) PCT Filed: May 6, 2021
(86) PCT No.: PCT/IB2021/053853

§ 371 Date: Nov. 3, 2022

(30) Foreign Application Priority Data
May 7, 2020 (IT) 102020000010228

(51) Int. Cl. A61K 31/714; A61K 33/30; A61K 45/06; A61K 9/28; A61K 9/08; A61K 9/00

(52) U.S. Cl. CPC A61K 31/714; A61K 33/30; A61K 45/06; A61K 9/2866; A61K 9/08; A61K 9/0053

(57) ABSTRACT

A composition for oral use comprising folic acid or derivatives and precursors thereof, an alkalizing agent selected from the group consisting of Magnesium Oxide, Calcium Oxide, Zinc Oxide and mixtures thereof, and optionally Vitamin B12 or derivatives and precursors thereof. The composition is intended for use in the treatment and/or prevention of the disease states caused by deficiency of folate and/or vitamin B12, particularly hyperhomocysteinemia.

### FIELD OF THE INVENTION

The invention relates to a composition for oral use, intended for the prevention and/or treatment of the conditions caused by deficiency of folate and/or Vitamin B12, including, in particular, hyperhomocysteinemia.

### State of the Art

Vitamin B12 (Cyanocobalamin, Methylcobalamin) and Folic Acid (pteroyl(mono)-glutamic acid, vitamin B9, folacin) are two vitamin factors with a known biochemical and metabolic role.

Folic acid and folates are known as vitamin B9, and although the two terms are often used as synonyms, there is a difference:
folate refers to the vitamin in the natural form thereof present in foods; it can also be produced synthetically and is called methyltetrahydrofolate.
folic acid is the oxidized form of the vitamin, and is the synthetic molecule present in vitamin formulations and added to so-called fortified foods.

With respect to folic acid, methyltetrahydrofolate is quickly absorbed by our body and is immediately bioavailable.

Folic acid contributes to the methylation of DNA bases, as well as the methylation of the haeme group and homocysteine, converting it to Methionine. In fact, folic acid supplementation is commonly recommended during pharmacological treatment with antiblastic drugs and antimetabolites, such as methotrexate, whose selective inhibition activity of the enzyme Dihydrofolate Reductase is responsible for the depletion of folic acid with toxicological repercussions on the liver (alterations in trophism, elevation of transaminases), mucous membranes and Cysteine metabolism.

In recent decades, folic acid has been recognized as essential in preventing some congenital malformations, particularly neural tube defects (NTDs).

Low folate levels due to insufficient dietary intake or other conditions such as alcohol abuse, the onset of certain diseases such as insulin-dependent diabetes mellitus or coeliac disease—result in reduced production of red blood cells in the blood, resulting in anaemia.

In particular, in the context of amino acid metabolism, folate presides over the reaction of homocysteine remethylation with the transformation thereof into methionine. A deficiency of folic acid or folates blocks the transformation reaction in question, leading to an increase in homocysteine on the one hand and a lack of methionine synthesis on the other.

Folic acid is predominantly taken in the form of sodium salt or calcium salt, in the case of the folinic acid derivative thereof, which represents the dihydroreduced form thereof, particularly indicated in supplementation during therapy with methotrexate, an inhibitor of the enzyme dihydrofolate reductase which would interfere with the reduction step to dihydrofolate.

More recently, synthetic derivatives such as sodium or calcium 5-methyltetrahydrofolate (5-MTHF), and the most recent glucosamine salt, capable of reversing the effects of the mutation of the gene encoding methyltetrahydrofolatase (MTHFR), have been synthesized and commercially introduced.

Vitamin B12, in the form of the vitamer methylcobalamin, catalyses the transfer of a methyl from methyl-tetrahydrofolate to homocysteine, to transform it into methionine.

Introduced with some foods such as meat, liver, eggs, milk, grains, some vegetable sources, especially those with broad leaves, but also beans and various legumes, methionine results in the formation of homocysteine by the loss of a methyl group, through the activation of methionine to adenosylmethionine (SAMe), universal donor of the methyl group; once the methyl is ceded, SAMe produces adenosylcysteine which releases homocysteine and adenosine by hydrolysis. The homocysteine can in turn be transformed back into methionine by a remethylation process. This methionine sparing reaction is catalysed by the enzyme methionine synthetase (MS) which requires 5 methyl-tetrahydrofolate (MTHF) as substrate and methylcobalamin as cofactor to transfer the methyl group of MTHF to homocysteine: methionine and tetrahydrofolate (THF) are thus formed. The cycle tends to conserve methionine which, in the activated form (SAMe), is the largest methyl donor for DNA, RNA, etc. (FIG. 1).

There are several molecular forms of Vitamin B12, including Cyanocobalamin, Methylcobalamin, Hydroxocobalamin and 5′-deoxyadenosylcobalamin¹. Such forms differ by the substitution of a different radical coordinated with the Cobalt atom of the Cobalamin porphyrin nucleus (FIG. 2).

The metabolically active forms in vivo are Methylcobalamin and 5′-deoxyadenosylcobalamin. Cyanocobalamin is the substance formed by extraction from meat with pepsin (protease activated by cyanide groups); Cyanocobalamin represents a kind of pro-drug of Cobalamin, the most frequent form of Vitamin B12 in supplemental and pharmaceutical oral and injection products.

Taken with food, Vitamin B12 is transformed into the absorbable form thereof in the stomach by the following steps:
Separation from proteins (especially meat) to which it is bound, by the action of pepsin and hydrochloric acid;
Conjugation with cobalophylline, also called salivary polypeptide R binder or aptocorrin (AC), a glycoprotein secreted in saliva²;
Binding of Cobalamin to Castle Intrinsic Factor (FIC)², a glycoprotein secreted by parietal cells in the stomach.

The Cobalamin-AC-FIC complex reaches the small intestine where, in the terminal part thereof (Ileum), it is absorbed upon detachment from Cobalophylline via a pseudo-neutral pH activated protease; following endocytosis in the ileal enterocytes (FIG. 3), the Cobalamin-FIC complex is internalized and subsequently released from the FIC inside lysosomal vesicles².

The Cobalamin is thus free to leave the enterocyte for exocytosis with the distal enterocytic membrane, pouring into the circulatory stream into which it is transported by Transcobalamin (transport proteins).

Vitamin B12 deficiency is quite rare, and can only occur in cases of a strict vegetarian diet. Other clinical implications are assumption during pregnancy for the prevention of spina bifida in newborns, for the proper maturation of the neural tube and nerve trophism.

With different biochemical mechanisms, Vitamin B12 and Folic Acid promote the synthesis and functionalization of haeme and the synthesis of nucleic acids; in fact, both folic acid and Vitamin B12 are involved in the cell division processes, especially in the gestational, neonatal, growth age; in the trophism of rapidly differentiating and turnover tissues, such as labile epithelia and mucous membranes; in the turnover of haeme.

Both are specifically involved in the synthesis of haemoglobin, a globular haemoprotein with tetra-pyrrolic structure (proto-porphyrin) at the centre of which is a Cobalt atom coordinated with the four pyrrolic rings. This protein plays the role of transporting oxygen to tissues inside the erythrocytes in vertebrates.

Since Vitamin B12 and Folic Acid are involved in the correct synthesis of haemoglobin, a deficiency thereof due to nutritional or dysfunctional reasons preludes an altered biosynthesis of this protein and the consequent altered transport of oxygen to the tissues which results in anaemia.

The main clinical manifestation of folate and Vitamin B12 deficiency is macrocytic megaloblastic anaemia, better known as pernicious anaemia.

As discussed above, it is further understood that the role of Vitamin B12 is closely related to that of folic acid in promoting DNA synthesis through the process of methylation and the conversion of homocysteine to methionine.

In this sense, another important clinical implication from folate and Vitamin B12 deficiency is hyperhomocysteinemia, consisting of an excessive concentration of homocysteine in the blood.

Homocysteine is an essential sulphur amino acid which is introduced into our body with food (proteins).

The metabolism thereof is regulated by the activity of enzymes and vitamins such as folates or folic acid and vitamins B6 and B12. A deficiency of folate/folic acid and these vitamins can cause homocysteine to accumulate, damaging the walls of blood vessels. When plasma homocysteine levels reach concentrations that are too high, i.e., above 12 μmol/L, hyperhomocysteinemia is referred to.

High levels of this amino acid negatively affect the nervous, cardiovascular and bone system functions, particularly through an increase in free radical production and the oxidative stress which results. For this reason, hyperhomocysteinemia is considered a risk factor for cardiovascular diseases and for the occurrence of neurological diseases such as cognitive decline and Alzheimer's disease.

Problems of the Background Art

Folates contained in the diet in the form of polyglutamates must be previously reduced to monoglutamate folic acid by an enteric reductase, to be absorbed at the ileal enterocyte level.

Despite the various molecular forms of folic acid developed to date, aimed at increasing the absorption thereof even in the case of transient or permanent anomalies of the biochemical systems capable of making it bio-accessible, substantial evidence remains that folic acid, in contact with gastric fluid, will be in its non-ionized form of carboxylic acid, a form in which the water solubility of the same is reduced very significantly (<0.01 mg/ml); in fact, gastric fluid has a notoriously variable pH between 1.5 and 2.0 on an empty stomach and between 3.5 and 4.5 on a full stomach. This will occur regardless of its pre-hydrogenated folinic acid form and the salts thereof, or pre-hydrogenated and pre-methylated 5-MTHF and the soluble salts thereof.

Such a loss of solubility results in the almost total precipitation of the same in the gastric fluid, a fact which itself makes much more difficult the transit of the molecule to the pylorus³ and its subsequent pouring into the enteric fluid where the pH rises to 6.8, a value that makes folic acid almost completely ionized and therefore free to come into contact with the ileal enterocytes where it is absorbed.

Such a problem is very often solved with the use of enteric-coated pharmaceutical forms, which convey and release folic acid directly at the intestinal level.

However, this formulation choice is responsible for a second problem, linked to the forms of composite supplementation of folic acid and B complex vitamins which, as mentioned, are involved in the same physiological processes in which folic acid intervenes and therefore have a complementary role to the latter.

In this sense, the supplemental forms of Vitamin B12, in the form of tablets, granules or liquid forms, in the presence of folic acid, and sometimes, also of other B complex vitamins, can be ineffective in promoting the absorption of the same at the enterocyte level, mainly due to an altered uptake of Vitamin B12 with salivary AC and FIC caused by “enteric coated” or gastro-protective forms which prevent or drastically reduce such a phenomenon.

### SUMMARY OF THE INVENTION

The Applicant has now developed a new composition, suitable for conveying folic acid, which solves the problems of the prior art.

The object of the present invention is a composition for oral use comprising folic acid or the derivatives and precursors thereof, in association with an alkalizing agent selected from the group consisting of Magnesium Oxide, Calcium Oxide, Zinc Oxide and mixtures thereof, where Zinc Oxide is preferred among those mentioned.

It should be noted that such a composition can advantageously be combined with Vitamin B12 for the prevention or treatment of disease states caused by folate and/or Vitamin B12 deficiency, preferably hyperhomocysteinemia.

A further object of the present invention is mouth-dispersible or swallowable solid formulations comprising the composition of the invention.

### Advantages of the Invention

The technical problem related to the precipitation of folic acid at gastric level, under fasting conditions, is solved by the Applicant through the use of alkalizing agents capable of raising the gastric pH to values suitable for the local dissolution of folic acid and maintaining it as such for a sufficient time to empty the fasting stomach.

Once dissolved in salivary fluid and swallowed or disaggregated in gastric fluid, by hydrating metal oxide, the composition promotes the partial neutralization of gastric hydrochloric acid in a pH range of 4.5 to 5.5, so as to create the conditions for an ionization of folic acid such as to make it soluble in fasted-state gastric fluid, preventing the precipitation thereof.

Under these pH conditions, completely similar to those of the full stomach (fed state), folic acid can dissolve and at the same time avoid being retained or adsorbed in the chyme.

The composition of the invention further allows to associate Vitamin B12 and folic acid without incurring the problems of inadequate absorption of the compositions of the prior art; in fact, the solution to the technical problem proposed by the Applicant favours the assimilation of Vitamin B12 and folic acid from oral pharmaceutical forms, regardless of the different chemical forms thereof. Therefore, the composition is particularly suitable for the treatment of hyperhomocysteinemia and, more generally, the increased need for Vitamin B12 and folates. Finally, it should be noted that the possibility of conveying other active ingredients in the composition of the invention makes it particularly suitable even in subjects with an increased need for other anti-anaemic substances and promoters of erythropoiesis.

Such a composition allows an effective conjugation of Vitamin B12 with aptocorrin (AC), secreted in saliva and poured into the stomach, in order to guarantee the physiological process which preludes enteric absorption and in particular the formation of the Vit B12-AC-FIC complex (Castle Intrinsic Factor).

Furthermore, the use of Zinc Oxide, in addition to promoting the dissolution of folic acid like other alkalizing agents, promotes the formation of Zinc Chloride by reaction with gastric hydrochloric acid, a bioavailable source of Zinc which will contribute to the reduction of homocysteinemia^{4,5}.

### DESCRIPTION OF THE FIGURES

FIG. 1: Methionine cycle.

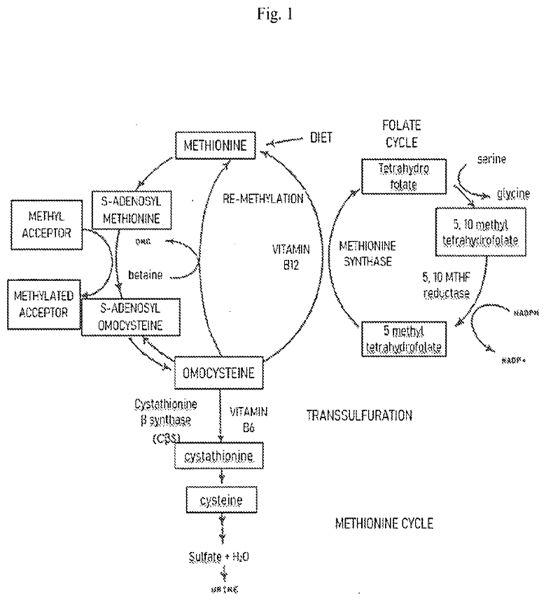

FIG. 2: Structural Cobalamin core. With R=—CH3 Methylcobalamin, with R=—CN, Cyanocobalamin, with R=-deoxyadenosyl, 5-deoxyadenosylcobalamin.

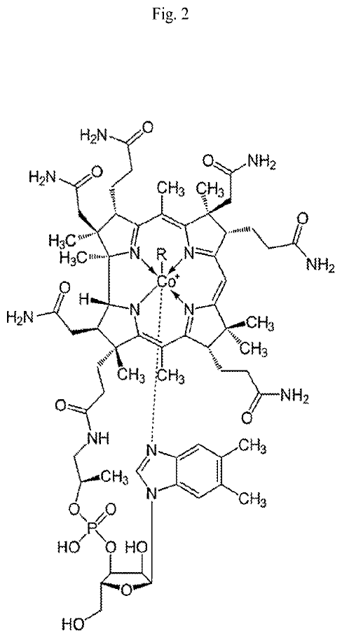

FIG. 3: Diagram of Cobalamin absorption in the intestine.

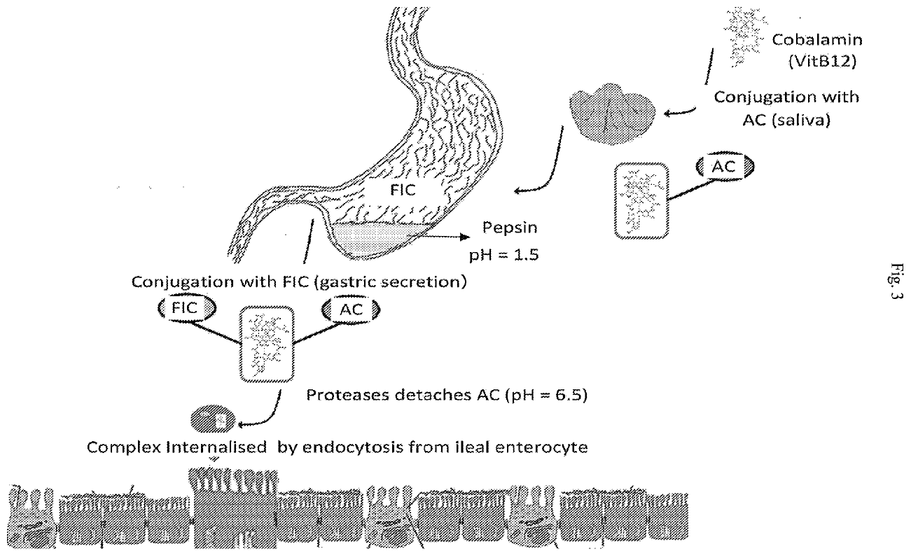

### DETAILED DESCRIPTION OF THE INVENTION

As mentioned above, the object of the invention is a composition for oral use comprising, as an active ingredient, folic acid or the derivatives and precursors thereof, and as an alkalizing agent, a metal oxide selected from the group consisting of Magnesium Oxide, Calcium Oxide, Zinc Oxide and mixtures thereof.

According to a preferred embodiment, the alkalizing agent is Zinc Oxide.

It should be noted that, preferably, the folic acid derivatives and precursors are selected from the group consisting of: salts of folic acid, folinic acid or its salts, 5-methyltetrahydrofolate (MTHF) or its salts, and mixtures thereof.

According to a preferred embodiment, the folic acid salts are selected from sodium salt and calcium salt.

According to a preferred embodiment, the folinic acid salts are selected from sodium salt and calcium salt.

According to a preferred embodiment, the salts of 5-methyltetrahydrofolate (MTHF) are selected from sodium salt, calcium salt and glucosamine salt.

An important aspect of the present invention is the use of oxides and not of the hydrated forms thereof as alkalizing agents; in fact, hydroxides are known alkalizing agents, characterized by a more vigorous neutralizing activity, immediate but not modulable over time.

The unhydrated form of the oxides has the advantage of allowing a controlled formation of the corresponding hydrates when the composition comes into contact with gastric fluids; this “controlled release” of the hydrates allows to raise the pH at the gastric level (pH between 4.5 and 5.5 for at least 60 minutes) to values compatible with the solubilization of folic acid or the derivatives and precursors thereof; it should be noted that such an alkalinization effect would not be possible, for such a prolonged period, using the corresponding hydroxides of the alkalizing agents indicated above.

It should be noted that preferably the composition is suitable for use as a medicament (medicinal specialty), nutraceutical, food supplement or food for special medical purposes.

According to a preferred embodiment, the oral composition is intended for use in the prevention and/or treatment of disease states caused by folate deficiency; it should be noted that, for the purposes of the present invention, hyperhomocysteinemia falls within the definition of folate deficiency disease state.

As already mentioned above, the use of antiblastic drugs or antimetabolites is sometimes responsible for a depletion of folic acid; in this sense, antiblastic or antimetabolic drug therapies induce a folate deficiency disease state.

The composition is therefore preferably suitable for use in association with antiblastic drug therapies or antimetabolites, preferably in association with therapeutic agents inhibiting Dihydrofolate Reductase, preferably in association with Methotrexate.

It should be noted that folate deficiency disease states can also occur in the event of inadequate diets or intestinal malabsorption; folic acid deficiency in the early stages of pregnancy significantly increases the risk of foetal malformations, in particular neural tube defects (NTDs). Furthermore, folate deficiency can be associated with other adverse pregnancy outcomes (intrauterine growth retardation, premature delivery).

In this sense, the composition of the invention is suitable for use in the prevention of neural tube defects during pregnancy.

The neural tube is an embryonic structure from which the central nervous system develops (brain, braincase, spine, etc.). When the neural tube does not close properly and completely during the first weeks of pregnancy, the newborn develops severe congenital malformations known as neural tube defects (NTDs).

It should be noted that NTDs are preferably selected from the group consisting of: spina bifida, anencephaly and encephalocele.

Spina bifida is due to an incomplete closure of the underside of the neural tube. Spina bifida also has very different consequences, ranging from problems which can be corrected with surgery to serious physical and mental disabilities. In the latter case, paralysis of the lower limbs, difficulty controlling the internal organs (intestine and bladder), developmental and learning difficulties and mental retardation, sometimes hydrocephalus, can occur. Children with spina bifida most often survive to adulthood.

Anencephaly is a condition in which the brain develops incompletely or does not develop at all as a result of incomplete closure of the upper part of the neural tube. Children with anencephaly die before or immediately after birth.

Encephalocele is a condition in which a part of the brain, more or less severely malformed, forms a hernia from a skull closure defect. Encephalocele can have an unfortunate outcome and only in a limited percentage of cases does normal psychomotor development occur⁶.

According to a preferred embodiment, the alkalizing agent and the folic acid or derivatives/precursors thereof are in a weight ratio between 20:1 and 200:1, preferably between 50:1 and 100:1, preferably between 65:1 and 90:1, preferably between 75:1 and 90:1, preferably between 78:1 and 88:1

It should be noted that, preferably, the amount of folic acid or the derivatives and precursors thereof per dosage unit is between 100 and 5000 μg, preferably between 200 and 1000 μg, preferably between 200 and 800 μg, preferably between 200 and 400 μg, preferably between 300 and 400 μg.

It should be noted that, preferably, the amount of alkalizing agent per dosage unit is between 20 and 100 mg, preferably between 20 and 80 mg, preferably between 20 and 60 mg, preferably between 25 and 50 mg.

It should be noted that, with particular reference to Zinc Oxide as an alkalizing agent, the maximum daily amount administered is 25 mg (corresponding to about 15 mg Zinc).

According to a preferred embodiment, the composition for oral use further comprises Vitamin B12 or derivatives/precursors thereof; this embodiment is particularly suitable for use in the prevention and/or treatment of disease states caused by folate and/or Vitamin B12 deficiency.

Preferably, the disease states caused by folate deficiency and/or Vitamin B12 are selected from the group consisting of hyperhomocysteinemia, anaemias and polyneuritis; it should be noted that the composition for oral use is particularly suitable for the prevention and/or treatment of hyperhomocysteinemia.

Preferably, the oral composition is intended for the prevention and/or treatment of macrocytic megaloblastic anaemia (pernicious anaemia).

It should be noted that the preferred form of the oral composition of the invention comprises folic acid or derivatives and precursors thereof, Zinc Oxide as alkalizing agent and Vitamin B12 or derivatives and precursors thereof; the therapeutic uses for which such embodiment is intended are those described in the description of the present application, preferably hyperhomocysteinemia.

It should be noted that the derivatives and precursors of Vitamin B12 are preferably selected from the group consisting of: Cyanocobalamin, Methylcobalamin, 5′-deoxyadenosylcobalamin, hydroxocobalamin and mixtures thereof.

According to a preferred embodiment, the amount of Vitamin B12 or the derivatives and precursors thereof is between 1 and 2000 μs, preferably between 1 and 1000 μs, preferably between 5 and 1000 μs, preferably between 5 and 500 μs, preferably between 5 and 100 μs, preferably between 5 and 50 μg.

Preferably, the oral composition is made in solid formulation form selected from tablets and granules. Preferably, the solid formulations suitable for conveying the composition are selected from, for example, swallowable tablets, two-layer tablets, filmed tablets, mouth-dispersible tablets, coated granules and mouth-dispersible granules.

According to an alternative embodiment, the composition for oral use is prepared in the form of a liquid formulation based on hydro-polyol; preferably, the liquid formulations suitable for conveying the composition are for example drops or syrups.

According to a first particularly preferred embodiment, the oral composition is made in the form of a mouth-dispersible solid formulation, preferably a granulate or a tablet, comprising a core and a mouth-soluble coating, which envelops the core externally. In fact, such an embodiment, allows to convey the folic acid or the derivatives and precursors thereof, the alkalizing agent and Vitamin B12 or the derivatives and precursors thereof in the outer coating, so as to allow the first active ingredient a correct solubilization at the gastric level, by virtue of the presence of the alkalizing agent; the second active ingredient is allowed a correct conjugation with AC at the salivary level, with FIC at the gastric level, thus ensuring a correct absorption in the subsequent intestinal transit.

According to a second particularly preferred embodiment, the composition for oral use is prepared in the form of a swallowable tablet which rapidly disaggregates in gastric fluid; said swallowable tablet comprises a core which disaggregates into gastric fluid, and an outer coating which completely covers said core. It should be noted the disaggregation rate of the core can be modulated with the use of suitably selected excipients, such as Polyvinylpolypyrrolidone (PVPP), so as to allow the disaggregation thereof in the gastric fluid, on an empty stomach, preferably within 10 minutes; the coating can be selected from the traditional coating forms, known in the literature (Part 5, Chapter 46, “Coating of pharmaceutical dosage forms”, from “The Remington, The science and Practice of Pharmacy”, 21st edition—Lippincott Williams & Wilkins), such as coatings aimed at masking an unpleasant taste.

According to an alternative embodiment, the swallowable, rapid gastric disaggregation tablet is formulated so that the disaggregating core contains folic acid and alkalizing agent, preferably Zinc Oxide, while the outer coating contains Vitamin B12.

The rapid gastric disaggregating swallowable tablet allows to produce a partial neutralization of gastric pH resulting in ionization of folic acid or its derivatives and precursors, while ensuring a release of Vitamin B12 such that it can be conjugated with AC and FIC.

It should be noted that the core of such solid formulations described above can accommodate other active agents.

According to a preferred embodiment, the composition for oral use further comprises other active agents selected from the group consisting of: other B group vitamins, L-Methionine, S-Adenosylmethionine, Betaine, Choline, Iron at oxidation state 2+ and/or 3+ in the form of organic and inorganic salts, and mixtures thereof.

Such additional active agents can be conveyed into the core of the aforementioned oral solid dosage form of the mouth-dispersible or swallowable type.

It should be noted that such additional active agents can also be formulated, in the composition for oral use of the invention, in a slow release technological form.

According to a preferred embodiment, the composition for oral use also comprises excipients known to those skilled in the art for the manufacture of solid or liquid pharmaceutical forms for oral consumption (according to techniques known to those skilled in the art, as available in the literature (Part 5, chapters “Solutions Emulsions Suspensions and Extracts” and “Oral Solid Dosage Forms” of “Remington: The Science and Practice of Pharmacy”, David B. Troy, Paul Beringer, Lippincott Williams & Wilkins, 2006).

Merely by way of example, the excipients suitable for the manufacture of the composition object of the present invention are indicated below:
filling agents such as corn starch, microcrystalline cellulose mannitol, sucrose, sorbitol, lactose, maltodextrins,
gliding agents such as colloidal silica, precipitated amorphous silica, magnesium oxide and carbonate;
lubricating agents such as magnesium stearate;
disaggregating agents such as carboxymethylcellulose sodium salt (CMC Na), polyvinylpolypyrrolidone (PVPP);
colouring and flavouring agents;
hardness modulators such as sucrose, calcium phosphate;
coating agents such as shellac;
enteric coated modified release polymers such as methacrylic acid derivatives, hydroxypropylmethylcellulose, sodium alginate, crosslinked sodium carboxymethylcellulose;
polyols such as sorbitol, mannitol, erythritol, xylitol, glycerol,
pH correctors such as citric acid, ascorbic acid.

### Experimental Part

Gastric Simulated Fluid (GSF) Test.

For all the experiments, physiological gastric conditions were reproduced taking into account the volume of gastric fluid on an empty stomach (35-40 mL) (Mudie D M et al. Quantification of gastrointestinal liquid volumes and distribution following a 240 mL dose of water in the fasted state. Mol Pharm. 2014 Sep. 2; 11(9):3039-47) and the fact that in the stomach, the salt concentration is different from that of blood plasma (0.25% vs 0.9%) (Sandra Klein. The Use of Biorelevant Dissolution Media to Forecast the In Vivo Performance of a Drug. AAPS J. 2010 September; 12(3): 397-406).

The effect of the alkalizing agent on gastric pH was simulated in vitro by preparing a 0.2% saline solution of NaCl and bringing the pH of the solution to around 1.2-2 with 37% hydrochloric acid diluted 1:5.

GSF was prepared using the components in table 1, all tests were performed simulating body temperature (37° C.) and performed in triplicate.

TABLE 1
Simulated gastric fluid (GSF) composition

| GSF composition | |
|---|---|
| pH 1.2-2 | Quantity |
| NaCl | 0.2 |
| 37% HCl diluted 1:5 | as needed up to pH 1.2-1.5 |
| H₂O | as needed up to 100 |

400 ml of GSF was stirred with magnetic stirrer and settled at 37° C. and the three-point digital pH meter was applied. 40 mg of Folic Acid was added to this solution.

The bright orange dispersion was stirred for 5 minutes at 200 rpm and then the stirring was stopped.

After 10 minutes, the complete precipitation of folic acid was observed at the bottom of the beaker, demonstrating the poor solubility thereof under the simulated body conditions.

The same experiment was repeated adding 30 mg of Zinc Oxide to the same amount of folic acid. The system was allowed to stir for 5 minutes at 200 rpm and then stirring was stopped, recording a pH of 5.2 and the formation of a translucent orange solution in the absence of precipitation on the bottom, a sign of the dissolution thereof. Such a pH was maintained in the range 5.0-5.5 also by simulating physiological acid secretion, combining 2 ml of 0.1 M HCl every 15 minutes for a total of 60 minutes.

In fact, Zinc Oxide prevents the lowering of the physiological pH thanks to a progressive formation mechanism of Zinc hydroxide which opposes acidification.

### EXAMPLES

By way of non-limiting illustration, two examples of the inventive composition are given below.

1. Example of a Mouth-Dispersible Tablet Coated Externally with a Mouth-Soluble Film

| | |
|---|---|
| Outer film | |
| Sodium Folate | equal to 400 μg Folic Acid |
| Cyanocobalamin | 5.0 μg |
| Zinc Oxide | 35 mg |
| Sucralose | 1.0 mg |
| Vanilla flavouring | as needed |
| iron oxide yellow (E172) | as needed |
| iron oxide red (E172) | as needed |
| Hypromellose | as needed |
| Glycerol | as needed |
| Demineralized water | as needed |
| Core | |
| Microcrystalline cellulose | as needed |
| Calcium phosphate | as needed |
| Com starch | as needed |
| Betaine | 250 mg |
| Vitamin B2 (phosphate salt) | equal to 2.4 mg Riboflavin |
| Vitamin B6 | 3 mg |
| Precipitated amorphous silica | 3% w/w |
| Magnesium Stearate | 2% w/w |
| Carboxymethylcellulose | 2% w/w |

1.1 Physical Chemical Parameters:

Weight (average 50 tablets): 1.050 g

Hardness (in Kg average 50 tablets): 12

1.2 Preparation Method

Outer Film

Dissolve the Vitamin B12, sodium folate and sucralose in water and then disperse in the order Glycerin, hypromellose, vanilla flavouring, iron oxide red and yellow, zinc oxide under mechanical stirring 400 rpm until a homogeneous orange-red mixture is obtained.

Core

Weigh together Vitamins B2 and B6, half of the starch, cellulose and mix the powders together with a mechanical V-stirrer for at least 5 minutes. Separately combine the Betaine hydrochloride with the other half of the corn starch, silica and magnesium stearate and mix the powder mixture for at least 5 minutes. Combine the powder mixture with the vitamins with the one containing Betaine and stir the mixture in a V-stirrer for another 5 minutes. Sieve the powdered mixture with 80 mesh manual metal sieve. Proceed to compress the thus obtained powder in a semi-automatic tablet press. Proceed to film the cores thus obtained with the aqueous dispersion previously set up in the coating pan.

2. Example of Oral Solution

| | |
|---|---|
| Sodium Folate | equal to 33.90 mg Folic acid |
| Cyanocobalamin | 42.4 mg |
| Zinc Oxide | 2.65 g |
| Sucralose | 120.0 mg |
| Orange flavouring | as needed |
| Lemon flavouring | as needed |
| Glycerol | 74 g |
| Demineralized water | as needed up to 100 g |

2.1 Chemical-Physical Parameters:

pH=8.5

density (measured with digital densimeter): 1.18

average weight 20 gtt=1.20 g

Folic acid for 20 gtt=400 μg

Cyanocobalamin for 20 gtt=500 μg

Zinc for 20 gtt=21 mg

2.2. Preparation Method:

Weigh the Glycerin and disperse the Zinc Oxide, stirring with a mechanical stirrer at 300 rpm until a homogeneous and translucent dispersion is obtained. Dissolve in the order sodium folate, Vitamin B12 and Sucralose in the available water. Combine the aqueous solution with the glycerol dispersion, maintaining stirring until a fluid, translucent mixture is obtained. Finally, combine the flavourings and keep stirring for at least 5 minutes.

Divide the dispersion into 12 ml dropper bottles.

### REFERENCES

1. Bernhard Kräutler Biochemistry of B12-cofactors in Human Metabolism. Subcell Biochem, 56, 323-46 2012
2. Renata Kozyraki, Olivier Cases. Vitamin B12 Absorption: Mammalian

Physiology and Acquired and Inherited Disorders. Biochimie, 95 (5), 1002-7. May 2013
3. Younis I R, Stamatakis M K, Callery P S, Meyer-Stout P J. Influence of pH on the dissolution of folic acid supplements. Int J Pharm. 2009 Feb. 9; 367(1-2):97-102.
4. Esfandiar Heidarian, Massoud Amini, Mahmoud Parham, and Ashraf Aminorroaya. Effect of Zinc Supplementation on Serum Homocysteine in Type 2 Diabetic Patients with Microalbuminuria. Rev Diabet Stud. 2009 Spring; 6(1): 64-70.
5. Pakfetrat M, Shahroodi J R, Zolgadr A A, Larie H A, Nikoo M H, Malekmakan L. Effects of zinc supplement on plasma homocysteine level in end-stage renal disease patients: a double-blind randomized clinical trial. Biol Trace Elem Res. 2013 June; 153(1-3):11-5.
6. https://www.epicentro.iss.it/acido-folico/

1. An oral composition comprising
folic acid or derivatives and precursors thereof, and
an alkalizing agent selected from the group consisting of Magnesium Oxide, Calcium Oxide, Zinc Oxide, and mixtures thereof.

2. A method of treatment of conditions caused by folate deficiency, said method comprising
orally administering a composition comprising
folic acid or derivatives and precursors thereof; and
an alkalizing agent selected from the group consisting of Magnesium Oxide, Calcium Oxide, Zinc Oxide and mixture thereof.

3. The method according to claim 2, wherein the condition caused by folate deficiency is hyperhomocysteinemia.

4. The method according to claim 2, wherein the composition comprising
folic acid or derivatives and precursors thereof; and
an alkalizing agent selected from the group consisting of Magnesium Oxide, Calcium Oxide, Zinc Oxide and mixture thereof is administered in combination with antiblastic drug therapies or antimetabolites.

5. A method of prevention of foetal neural tube defects comprising the oral administration, during gestation, of a composition comprising
folic acid or derivatives and precursors thereof; and
an alkalizing agent selected from the group consisting of Magnesium Oxide, Calcium Oxide, Zinc Oxide, and mixtures thereof.

6. The oral composition according to claim 1, wherein the alkalizing agent and folic acid or derivatives and precursors thereof are in a weight ratio between 20:1 and 200:1.

7. The oral composition according to claim 1, wherein the folic acid derivatives and precursors are selected from the group consisting of: folic acid salts, folinic acid or salts thereof, 5-methyltetrahydrofolate (MTHF) or salts thereof, and mixtures thereof.

8. A method of treating diseases caused by deficiency of folate or Vitamin B12, said method comprising:
orally administering a composition comprising
folic acid or derivatives and precursors thereof;
an alkalizing agent selected from the group consisting of Magnesium Oxide, Calcium Oxide, Zinc Oxide and mixtures thereof; and
Vitamin B12 or derivatives/precursors thereof.

9. The method of treatment according to claim 8, wherein the disease states caused by deficiency of folate or Vitamin B12 are selected from the group consisting of: anemias, and polyneurites.

10. The oral composition according to claim 1, further comprising Vitamin B12 or derivatives/precursors thereof, wherein said derivatives/precursors are selected from the group consisting of: Cyanocobalamin, Methylcobalamin, 5′-deoxyadenosylcobalamin, hydroxycobalamin and mixtures thereof.

11. The oral composition according to claim 1, in the form of
solid formulation selected from the group consisting of: tablets and granulates; or
hydro-polyol-based liquid formulation.

12. The oral composition according to claim 1, further comprising other active ingredients selected from the group consisting of: other B vitamins, L-Methionine, S-Adenosylmethionine, Betaine, Choline, Iron at oxidation state 2⁺ or 3⁺ in the form of organic or inorganic salts, and mixtures thereof.

13-14. (canceled)

15. The oral composition according to claim 1, wherein said alkalizing agent is Zinc Oxide.

16. The method according to claim 2, wherein said alkalizing agent is Zinc Oxide.

17. The method according to claim 5, wherein said alkalizing agent is Zinc Oxide.

18. The method according to claim 8, wherein said alkalizing agent is Zinc Oxide.
